(19) United States
(12) Patent Application Publication
(10) Pub. No.: US 2009/0240244 A1
(43) Pub. Date: Sep. 24, 2009

(54) Electrosurgical Generator Having Boost Mode Control Based on Impedance

(75) Inventors: Jerry L. Malis, King of Prussia, PA (US); Anthony John Groch, Mantua, NJ (US)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: Synergetics USA, Inc., King of Prussia, PA (US)

(21) Appl. No.: 12/364,178

(22) Filed: Feb. 2, 2009

Related U.S. Application Data
(60) Provisional application No. 61/037,794, filed on Mar. 19, 2008.

(51) Int. Cl. A61B 18/18; A61B 18/12

(52) U.S. Cl. 606/33; 606/34

(57) ABSTRACT

A method of controlling output power of an electrosurgical generator apparatus that controls a variable output signal to a pair of electrodes includes setting the output power of the generator apparatus to a selected power output level. An impedance is measured across the electrodes when the electrodes are applied to an area of tissue. The output power of the generator apparatus is changed to a boost power output level greater than the selected power output level. The boost power output level corresponds to a calculation based at least in part on the measured impedance. The method further includes applying the output signal to the electrodes at the boost power output level for a first time duration and changing the power output to the selected power output level after the first time duration. An electrosurgical generator apparatus operating in accordance with the method is also described.

### CROSS REFERENCE TO RELATED APPLICATIONS

This application claims the benefit of U.S. Provisional Patent Application No. 61/037,794, filed on Mar. 19, 2008, entitled “Electrosurgical Generator Having Boost Mode Control Based on Impedance,” the entire contents of which are incorporated by reference herein.

### BACKGROUND OF THE INVENTION

An embodiment of the present invention relates generally to a method of providing a boost mode in an electrosurgical generator apparatus, and more particularly, to a method of providing a boost mode wherein the boost output power level is based on a measured impedance of tissue.

Devices used for controlling monopolar and bipolar electrode tools are well known in the art. U.S. Pat. No. 5,318,563, the contents of which are incorporated by reference herein, relates to electrosurgical radio frequency (RF) generators. The electrodes in the prior art systems are used for cutting and coagulation of tissue. An RF current is generated between the electrodes and is applied to the tissue. Regarding bipolar tools in particular, cutting occurs by application of the concentrated RF current to destroy cells placed between the electrodes.

It is found, however, that when the electrodes are placed in contact with the body prior to activation, the output voltage of the RF amplifier is decreased. As a result, the cutting ability of the electrosurgical tool is hindered. One solution has been to provide a short, initial boost to the power output level of the generator upon activation of the electrosurgical tool. The brief power output surge is enough to overcome the impedance caused by the tissue to allow cutting to begin. After the surge, the power output level returns to normal and cutting proceeds in the typical fashion.

The general practice has been to set the boost voltage to a certain level and use the same level regardless of the conditions. This can lead to an increase in collateral damage in the tissue caused solely by the power surge. For example, the impedance of tissue between individuals may vary greatly, and even within the same individual, different tissues exhibit various impedance levels. The impedance is correspondingly proportional to an amount of cell destruction caused by the generator apparatus. Therefore, a constant boost voltage of, for example, 1100 V may cause more unintended damage in a patient or tissue with a lower impedance level than in a patient or tissue having a higher impedance level.

It is desirable to provide a method of generating a boost voltage in an electrosurgical generator apparatus while minimizing the collateral damage to surrounding tissue when the boost voltage is applied. It is further desirable to provide an electrosurgical apparatus that provides a variable boost voltage for minimizing collateral damage to surrounding tissue.

### BRIEF SUMMARY OF THE INVENTION

Briefly stated, an embodiment of the present invention comprises a method of controlling output power of an electrosurgical generator apparatus that controls a variable output signal to a pair of electrodes. The method includes setting the output power of the generator apparatus to a selected power output level. An impedance is measured across the electrodes using an impedance monitoring circuit when the electrodes are applied to an area of tissue. The output power of the generator apparatus is changed to a boost power output level greater than the selected power output level. The boost power output level corresponds to a calculation based at least in part on the measured impedance. The method further includes applying the output signal to the electrodes at the boost power output level for a first time duration. The power of the output signal applied to the electrodes is changed to the selected power output level after the first time duration.

Another embodiment of the present invention comprises an electrosurgical generator apparatus that controls a variable output signal to a pair of electrodes. The generator apparatus includes a controller for controlling the generator apparatus. An impedance monitoring circuit detects an impedance as measured across the electrodes when the electrodes are applied to an area of tissue. A memory stores predetermined values for calculating a boost power output level based at least in part on the measured impedance. The controller is configured to change a selected power output level to the boost power output level based at least in part on the measured impedance for a first time duration and change the boost power output level to the selected power output level after the first time duration.

### BRIEF DESCRIPTION OF THE DRAWINGS

The foregoing summary, as well as the following detailed description of preferred embodiments of the invention, will be better understood when read in conjunction with the appended drawings. For the purpose of illustration, there are shown in the drawings embodiments which are presently preferred. It should be understood, however, that the invention is not limited to the precise arrangements and instrumentalities shown.

FIG. 1A is an elevational view of a front panel of an electrosurgical generator apparatus in accordance with a preferred embodiment of the present invention;

FIG. 1B is an elevational view of a rear panel of the electrosurgical generator of FIG. 1A;

FIG. 2A is a perspective view of an electrosurgical bipolar instrument for use in accordance with the electrosurgical generator of FIG. 1A;

FIG. 2B is a perspective view of an electrosurgical monopolar instrument for use in accordance with the electrosurgical generator of FIG. 1A;

FIG. 3 is a control circuit block schematic diagram in accordance with a preferred embodiment of the present invention;

FIG. 4 is a screenshot from a display of an electrosurgical generator apparatus in accordance with a preferred embodiment of the present invention;

FIG. 5 is a flowchart depicting a method of supplying a boost power output level from an electrosurgical generator apparatus in accordance with a preferred embodiment of the present invention; and

FIG. 6 is a table of multipliers for determining a boost power output level stored in memory of an electrosurgical generator apparatus in accordance with a preferred embodiment of the present invention.

### DETAILED DESCRIPTION OF THE INVENTION

Certain terminology is used in the following description for convenience only and is not limiting. The words “right”, “left”, “lower”, and “upper” designate directions in the drawings to which reference is made. The words “inwardly” and “outwardly” refer to directions toward and away from, respectively, the geometric center of the apparatus and designated parts thereof. The terminology includes the above-listed words, derivatives thereof, and words of similar import. Additionally, the words “a” and “an”, as used in the claims and in the corresponding portions of the specification, mean “at least one.”

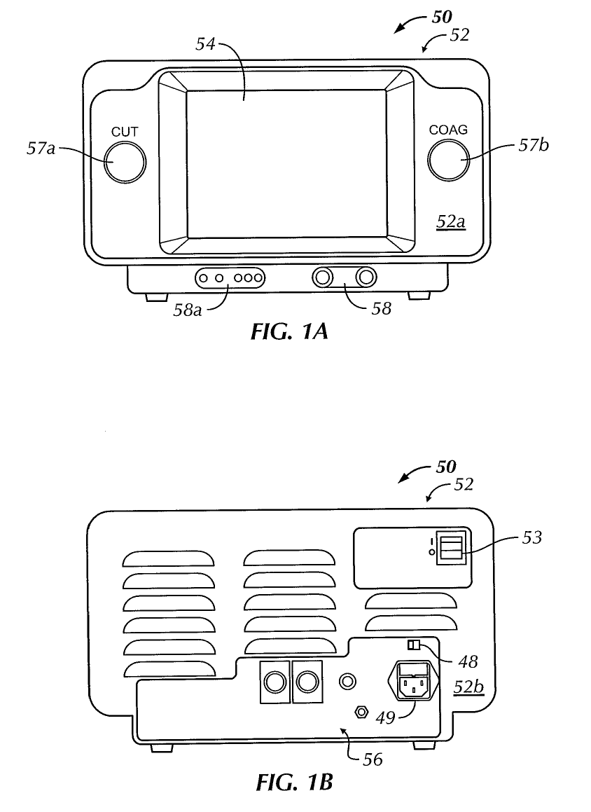

Referring to the drawings in detail, wherein like reference numerals indicate like elements throughout, there is shown in FIGS. 1A and 1B a preferred embodiment of an electrosurgical RF generator apparatus or RF generator 50. FIG. 1A is an elevational view of a front panel 52a of the RF generator 50, and FIG. 1B is a perspective view of a rear panel 52b of the RF generator 50.

The RF generator 50 includes a housing 52, a display screen 54, such as a cathode ray tube (CRT), liquid crystal display (LCD), or the like, on the front panel 52a and a connector panel 56 on the rear panel 52b. The display screen 54 is preferably a touch panel. Control knobs 57a, 57b on the front panel 52a may be used for selecting output power. A power cord (not shown) of the conventional type as is known in the art is connected to a power source to provide power to the RF generator 50 via a source power plug adapter 49. Preferably, the RF generator 50 is supplied with between about 110-125 volts of alternating current (VAC) at 60 Hertz (Hz) or about 220-240 VAC at 50 Hz, and may be selected using the voltage supply switch 48. But, other supply voltages and frequencies of AC voltage or other direct current (DC) voltages may be supplied without departing from the present invention. The RF generator 50 also includes an on/off power switch 53. The RF generator 50 may also include one or more speakers or audio outputs (not shown) for generating indicator beeps and/or vocal instructions in one or more selectable languages.

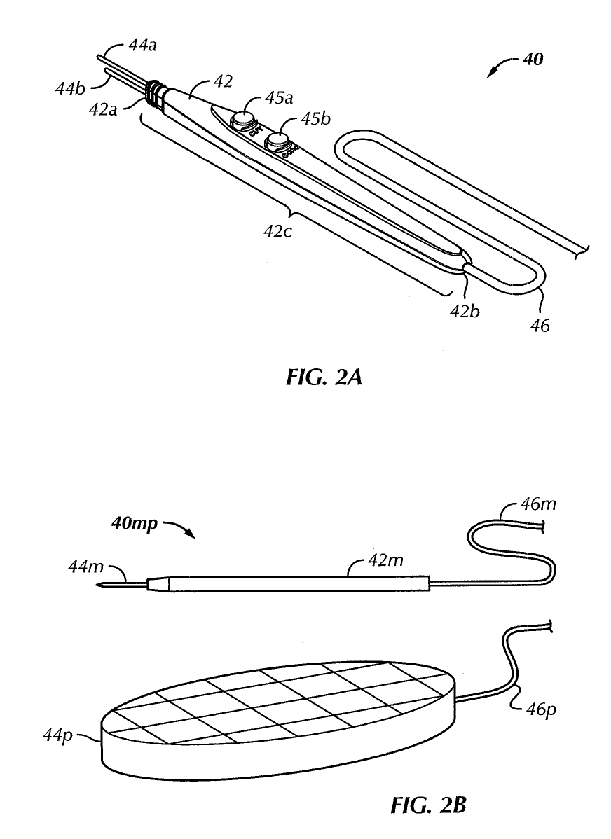

The RF generator 50 may be connected to either a monopolar electrosurgical tool (e.g., as shown in FIG. 2B) or a bipolar electrosurgical tool (e.g., as shown in FIG. 2A). Preferably, the RF generator 50 is used with a bipolar surgical pen 40, shown in FIG. 2A, having a cord 46 connected to an output adapter 58 (or alternate output adapter 58a) of the RF generator 50. The bipolar surgical pen 40 is well known in the art and typically includes an instrument housing 42 having a distal end 42a, a proximal end 42b, and an elongated body 42c therebetween. The cord 46 from the output adapter 58 of the RF generator 50 attaches to the surgical pen 40 at the proximal end 42b. First and second cut/coagulate mode push buttons 45a, 45b are located on the upper surface of the instrument housing 42. Alternatively, mode selection between cut and coagulate may be placed on the RF generator 50 or a foot pedal (not shown). A pair of RF electrodes 44a, 44b are located at the distal end 42a of the instrument housing 42. The electrodes 44a, 44b are each of opposite polarity such that one electrode is positively charged and the other electrode is negatively charged, alternately, during use. The electrodes 44a, 44b can be of varying sizes, shapes and thicknesses depending upon the particular application.

A monopolar electrosurgical tool 40mp is shown in FIG. 2B, and may alternatively be used with the RF generator 50. The monopolar electrosurgical tool 40mp comprises a pen 42m and an electrode pad 44p. A cord 46m of the pen 42m connects to the RF generator 50 through, for example, output adapter 58. An electrode 44m of the pen 42m is applied to the tissue of a patient. The electrode pad 44p is applied to the patient and is separately connected to the RF generator 50 via a cord 46p. For simplicity, the preferred embodiments will be described as using the bipolar surgical pen 40, but those skilled in the art will recognize that a monopolar electrosurgical tool 40mp may be substituted therefor.

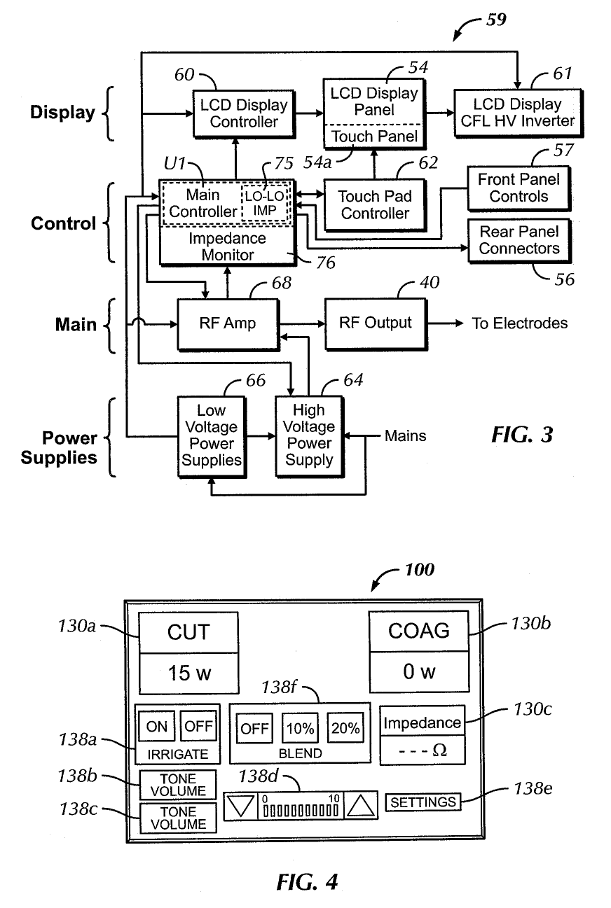

Referring to FIG. 3, an overall control circuit 59 for the RF generator 50 is shown in a general block diagram. The control circuit 59 is comprised of multiple sub-circuits forming an overall control system for the RF generator 50. The control circuit 59 includes a main controller U1 and high and low voltage power supplies 64, 66. Preferably, the RF generator 50 includes a high voltage (HV) power supply 64 that is an off-line switching power supply to provide a high voltage DC output to an RF amplifier circuit 68. The HV power supply 64 receives supply voltage (e.g., 120 VAC, 60 Hz) and serves as the power source for the RF amplifier 68. The touch panel 54a is controlled by an LCD or simply display controller 60 and is powered by an LCD or simply display compact fluorescent lamp (CFL) HV inverter 61. Inputs from the touchscreen 54a are interfaced through a touch pad controller 62. The touch pad controller 62 interfaces with the main controller U1. The front panel controls 57 and rear panel connectors 56 provide input/output (I/O) to the control circuit 59. The main controller U1 controls the RF amplifier circuit 68. The RF amplifier circuit 68, which serves to modulate a carrier signal, in combination with the HV power supply 64 provide a variable signal output to the bipolar surgical pen 40. Feedback from the bipolar surgical pen 40 may be sensed by an impedance monitor circuit 76.

The impedance monitor circuit 76 is connected in parallel with an RF output and filter of the RF amplifier 68. Impedance is thereby detected using the electrodes 44a, 44b of the surgical pen 40, and the actual impedance of the tissue to be cut or coagulated may be calculated. The impedance value is used by the main controller U1 to determine a boost voltage to apply at the initial cutting stage, as described in further detail below. The main controller U1 further includes a partial short circuit detection monitor 75, shown in FIG. 3 as a “low-low” impedance monitor. The partial short circuit detection monitor 75 detects partial shorts that significantly drop measured impedance levels that may result in boost elevations that may present safety hazards, or damage or melt the tips of the electrodes 44a, 44b. The partial short circuit detection monitor 75 is configured to limit boost current when the measured impedance is less than a predetermined or operator adjustable “low-low impedance” set point.

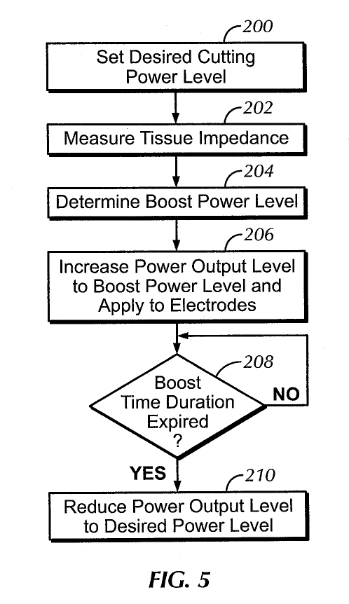

FIG. 4 is a screenshot 100 displayed on touchscreen 54a that may be shown during a typical cut mode of the RF generator 50. The screen 100 includes onscreen indicators 130a-130c for cut power output (130a), coagulate power output (130b), and measured impedance (130c). In particular, an operator may select the cutting power output of the RF generator 50 by adjusting the cut control knob 57a (FIG. 1A). Similarly, an operator may select the coagulating power output by adjusting the coagulate control knob 57b. An option panel 138a allows a user to select whether to irrigate the electrodes 44a, 44b during operation. Option panels for adjusting tone volume (138b) and voice volume (138c) are provided, wherein the user may adjust the volume level for either setting using the volume selector panel 138d. A settings menu for adjusting further parameters of the RF generator 50 is provided to the user upon selection of the settings button 138e. The RF generator 50 may also provide the user with an option to “blend” cutting and coagulation operations, selectable at various levels by a blend control panel 138f.

It will be appreciated by those skilled in the art that the RF generator 50 need not utilize a touchscreen 54a for displaying and selection of information. For example, selections may be made by an operator using conventional knobs, switches, or the like. Further, information may be conveyed to the operator using alphanumeric light emitting diode (LED), LCD, or other displays known in the art.

FIG. 5 is a flowchart illustrating a method in accordance with preferred embodiments of the present invention. At block 200, a desired cutting power output level is set. The desired power output level may be set manually by the operator by, for example, adjusting the control knob 57a. Alternatively, the desired power output level may be a predetermined value associated with the cutting mode. In any event, the desired power output level is typically the power output level for the cutting operation of tissue under normal conditions.

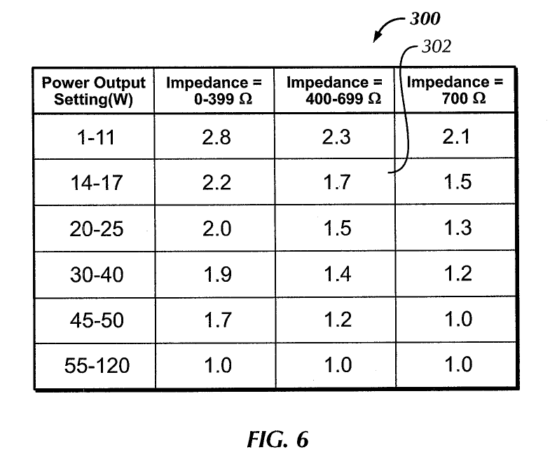

At block 202, when the electrodes 44a, 44b of the surgical pen are applied to an area of tissue, the impedance monitor circuit 76 measures an impedance. At block 204, the value of the measured impedance is used by the main controller U1 to determine a boost power output level that is greater than (or equal to) the desired power output level. In preferred embodiments, the controller U1 additionally accounts for the desired power output level and determines the boost power output level as a multiple of the desired power output level. For example, FIG. 6 shows a table 300 stored in a memory of the main controller U1. The table 300 considers the desired power output level and the measured tissue impedance and lists a number of multipliers associated with various combinations of the two values. For example, for a desired power output level of 15 W and a tissue impedance of 500 Ω, the main controller U1 proceeds to block 302 and retrieves a multiplier of 1.7. The multiplier is applied to the desired output level to obtain the boost power output level, or in this instance, 15 W×1.7=25.5 W. It is noted that under certain conditions several of the multipliers in the table 300 are listed as 1.0. For such conditions, the desired power output level is already sufficient to overcome the tissue impedance and no boost is required.

Returning to FIG. 5, having determined the boost power output level, the main controller U1 increases the power output to the boost level and when the operator sends a signal to begin cutting, for example via foot pedal, push button, or the like, the increased power output is applied to the electrodes 44a, 44b of the surgical pen 40. The output signal provided by the RF amplifier 68 may be a sine waveform. However, during a boost time duration t_{b}, the signal may have an amplitude that differs from the amplitude of the signal following the boost time duration t_{b}.

In preferred embodiments, other characteristics of the output signal supplied to the surgical pen 40 may additionally be altered. For example, the waveform supplied by the RF amplifier 68 during the boost time duration t_{b} may differ from the waveform supplied thereafter. A Malis waveform, described in U.S. Pat. No. 4,590,934, the contents of which are incorporated by reference herein, may be applied during the boost time duration t_{b}. Periodic damping, a distinctive feature of the Malis waveform, provides further protection from collateral damage to the tissue. Once the boost time duration t_{b} has expired, RF amplifier 68 may return to a sine waveform. The peak amplitude of both the first and second waveforms may differ. Other waveforms (such as, for example, an impulse waveform) or combinations thereof may be used in keeping with preferred embodiments of the present invention. Other preferred embodiments of the present invention may include combinations of the signal variations described above or other variations such as to wavelength, frequency, or the like.

The boost power output level is applied only for a short duration t_{b}, long enough to overcome the tissue impedance and begin the cutting procedure. Preferably the boost power output voltage is applied for t_{b}=200 ms. The main controller U1 at block 208 therefore determines whether the boost time duration t_{b} has expired. If not, the electrodes 44a, 44b continue to receive the boost power output from the RF generator 50. Once the boost time duration t_{b} has expired, at block 210 the power output level is reduced to the initial desired power output level and cutting thereafter proceeds in the normal fashion.

It will be appreciated by those skilled in the art that changes could be made to the embodiments described above without departing from the broad inventive concept thereof. It is understood, therefore, that the invention is not limited to the particular embodiments disclosed, but it is intended to cover modifications within the spirit and scope of the present invention as defined by the appended claims.

We claim:

1. A method of controlling output power of an electrosurgical generator apparatus that controls a variable output signal to a pair of electrodes, the method comprising:
(a) setting the output power of the generator apparatus to a selected power output level;
(b) measuring an impedance across the electrodes using an impedance monitoring circuit when the electrodes are applied to an area of tissue;
(c) changing the output power of the generator apparatus to a boost power output level greater than the selected power output level, the boost power output level corresponding to a calculation based at least in part on the measured impedance;
(d) applying the output signal to the electrodes at the boost power output level for a first time duration; and
(e) changing the power of the output signal applied to the electrodes to the selected power output level after the first time duration.

2. The method of claim 1, wherein the output signal applied to the electrodes during the first time duration has a first peak amplitude and the output signal applied to the electrodes after the first time duration has a second peak amplitude, the first peak amplitude being different than the second peak amplitude

3. The method of claim 2, wherein the output signal applied to the electrodes during the first time duration is of a first waveform and the output signal applied to the electrodes after the first time duration is of a second waveform, the first waveform being different than the second waveform.

4. The method of claim 3, wherein the first waveform is one of an impulse waveform, a Malis waveform, and a sine wave.

5. The method of claim 2, wherein the output signal applied to the electrodes is in the form of a sine wave.

6. The method of claim 1, wherein the calculation of the boost power output level is additionally based in part on the selected power output level.

7. The method of claim 1, wherein the first time duration is about 200 milliseconds.

8. The method of claim 1, further comprising:
(f) providing a partial short circuit detection monitor.

9. The method of claim 1, wherein the pair of electrodes form one of a monopolar electrosurgical tool and a bipolar electrosurgical tool.

10. An electrosurgical generator apparatus that controls a variable output signal to a pair of electrodes, the generator apparatus comprising:
(a) a controller for controlling the generator apparatus;
(b) an impedance monitoring circuit that detects an impedance as measured across the electrodes when the electrodes are applied to an area of tissue; and
(c) a memory for storing predetermined values for calculating a boost power output level based at least in part on the measured impedance,
the controller being configured to change a selected power output level to the boost power output level based at least in part on the measured impedance for a first time duration and change the boost power output level to the selected power output level after the first time duration.

11. The electrosurgical generator apparatus of claim 10, further comprising:
(d) a radio frequency (RF) waveform generator circuit that provides modulation of a carrier signal, the carrier signal directly affecting the variable output signal applied to the electrodes.

12. The electrosurgical generator apparatus of claim 11, wherein the RF waveform generator circuit provides a first waveform having a first peak amplitude during the first time duration and a second waveform having a second peak amplitude after the first time duration, the first peak amplitude being different than the second peak amplitude.

13. The electrosurgical generator apparatus of claim 12, wherein the first waveform is different than the second waveform.

14. The electrosurgical generator apparatus of claim 13, wherein the first waveform is one of an impulse waveform, a Malis waveform, and a sine wave.

15. The electrosurgical generator apparatus of claim 12, wherein the first and second waveforms are sine waves.

16. The electrosurgical generator apparatus of claim 10, wherein calculation of the boost power output level using the predetermined values is additionally based in part on the selected power output level.

17. The electrosurgical generator apparatus of claim 10, wherein the first time duration is about 200 milliseconds.

18. The electrosurgical generator apparatus of claim 10, wherein the controller includes a partial short circuit detection monitor.

19. The electrosurgical generator apparatus of claim 10, wherein the pair of electrodes form one of a monopolar electrosurgical tool and a bipolar electrosurgical tool.
